(19) United States
(12) Plant Patent Application Publication
(10) Pub. No.: US 2009/0038041 P1
(43) Pub. Date: Feb. 5, 2009

(54) Hibiscus 'Panama Bronze'

(76) Inventor: John Ruter, Tifton, GA (US)

Correspondence Address: DICKINSON WRIGHT PLLC, 38525 WOODWARD AVENUE, SUITE 2000, BLOOMFIELD HILLS, MI, 48304-2970, US

(21) Appl. No.: 11/881,930

(22) Filed: Jul. 30, 2007

(51) Int. Cl. A01H 5/00

(52) U.S. Cl. PLT/257

(57) ABSTRACT

Hibiscus ‘Panama Bronze’ is an ornamental variety, crossed and cultivated in Georgia. The plants of the new Hibiscus display an intense bronze color in high light, deeply cut foliage, stable foliage color, very large flowers, thrives in hot an humid conditions, and flowers heavily during short days (November to April) in zone 10. ‘Panama Bronze’ can be grown in the garden or in a container. Page 7 of 8

### LATIN NAME

Hibiscus ‘Panama Bronze’ is of the genus and species Hibiscus acetosella×Hibiscus radiatus.

### VARIETY DENOMINATION

The new Hibiscus claimed is of the variety denominated ‘Panama Bronze.’

### CROSS-REFERENCE TO RELATED APPLICATIONS

This patent application is related to co-pending application entitled Hibiscus ‘Panama Red,’ invented by the same Inventor, assigned to the same Assignee, and filed simultaneously with, the present application. Cross-referenced application Hibiscus ‘Panama Red’ is the maternal parent plant of the present Hibiscus ‘Panama Bronze.’

### BACKGROUND OF THE INVENTION

The present invention relates to a new and distinct cultivar of Hibiscus, botanically known as Hibiscus acetosella×Hibiscus radiatus, and herein referred to as ‘Panama Bronze.’

The new Hibiscus is a product of a planned breeding program conducted by the Inventor at the Tifton campus of the University of Georgia in Tifton, Ga. The objective of the Hibiscus breeding program is to create new plant cultivars with ornamental leaf distinctions and abundant flowers.

The new Hibiscus originated from a cross-pollination made by the Inventor during 2005, of the Hibiscus acetosella ‘Panama Red’ (co-pending patent application) as the female and Hibiscus radiatus (not patented) as the male, or pollen parent. The cultivar ‘Panama Bronze’ was discovered and selected by the Inventor as a flowering plant within the progeny of the stated cross-pollination in a controlled environment in 2006.

Asexual reproduction of the new Hibiscus by vegetative terminal cuttings in a controlled environment in Tifton, Ga. since 2006, has shown that the unique features of this new Hibiscus are stable and reproduced true to type in successive generations.

### BRIEF SUMMARY OF THE INVENTION

The cultivar ‘Panama Bronze’ has not been observed under all possible environmental conditions. The phenotype may vary somewhat with variations in environment and cultural practices such as temperature and light intensity without, however, any variance in genotype.

The following traits have been repeatedly observed and are determine to be the unique characteristics of ‘Panama Bronze:’
1. Bronze foliage color;
2. Deeply cut foliage;
3. Stable foliage color;
4. Very large flowers;
5. Flowers heavily during short days (November to April) in zone 10; and
3. Thrives in hot and humid conditions.

Plants of the new Hibiscus can be compared to plants of the female parent, the cultivar ‘Panama Red.’ Plants of the new Hibiscus differ from ‘Panama Red’ in the following characteristics:
1. Plants of the new Hibiscus display an intense bronze color in high light and a green color in low light, compared to the female parent that shows a nearly blackish-purple in high light and a rich, dark red in low light.
2. Plants of the new Hibiscus have flowers that are one third larger compared to the female parent.
2. Plants of the new Hibiscus have more deeply cut and elongated foliage compared to the female parent.
3. Plants of the new Hibiscus are less dense compared to the female parent.

Plants of the new Hibiscus can be compared to plants of the male parent, the Hibiscus radiatus. Plants of the new Hibiscus differ from Hibiscus radiatus in the following characteristics:
1. Plants of the new Hibiscus display an intense bronze color in high light and a green color in low light, compared to the male parent that shows green in high light and green in low light.
2. Plants of the new Hibiscus display darker and larger flowers than the male parent.

### BRIEF DESCRIPTION OF THE DRAWINGS

FIG. 1 is a photograph of full plant of ‘Panama Bronze.’

FIG. 2 is a photograph of a flower of ‘Panama Bronze’ showing the large, dark color.

FIG. 3 is a photograph of a leaf of ‘Panama Bronze’ showing the unique bronze color of the foliage.

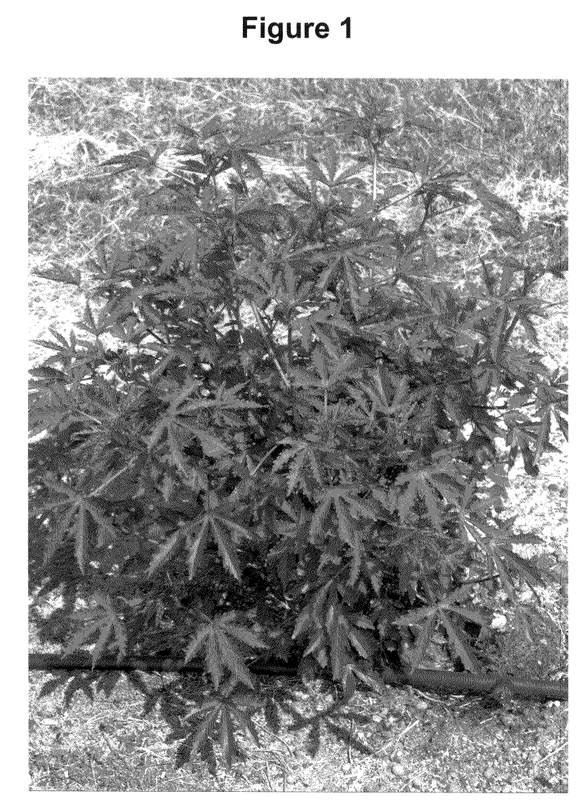

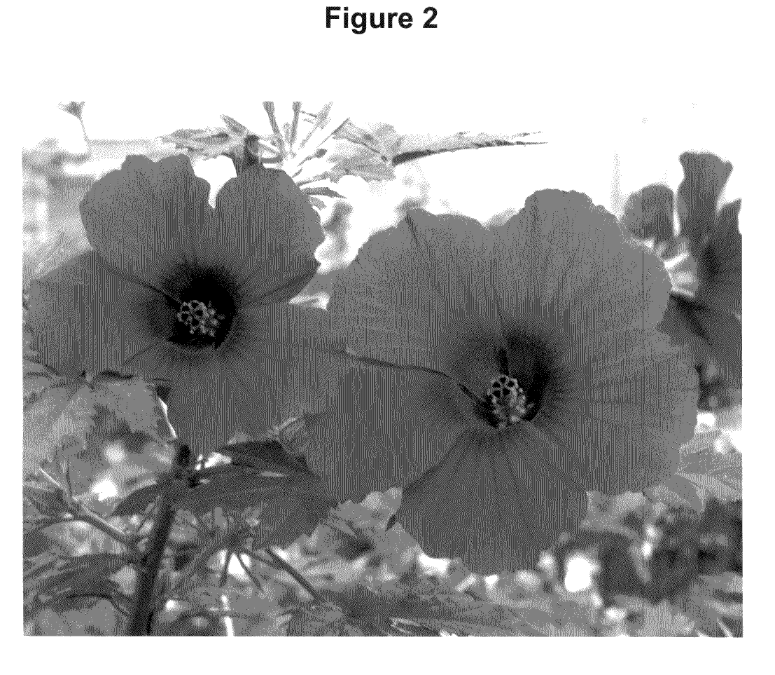

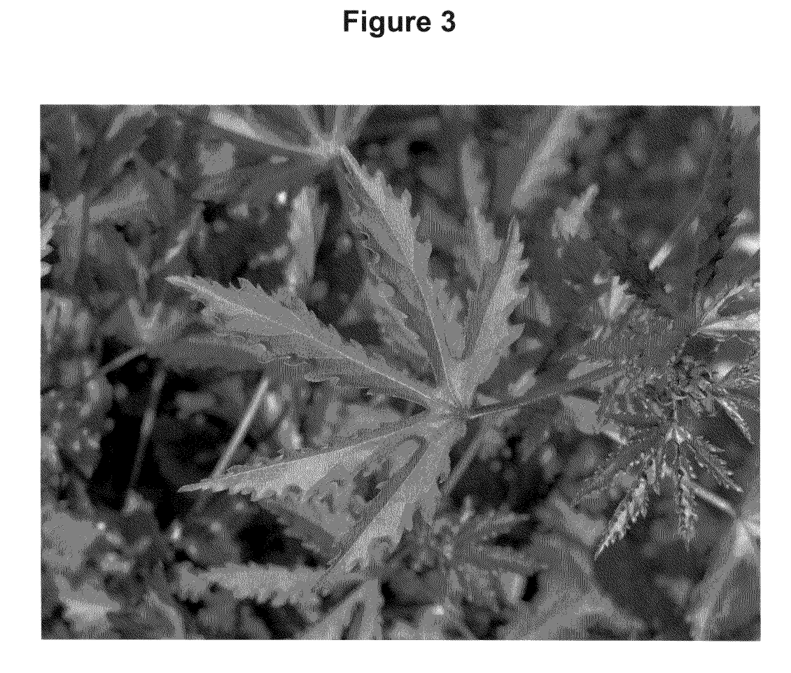

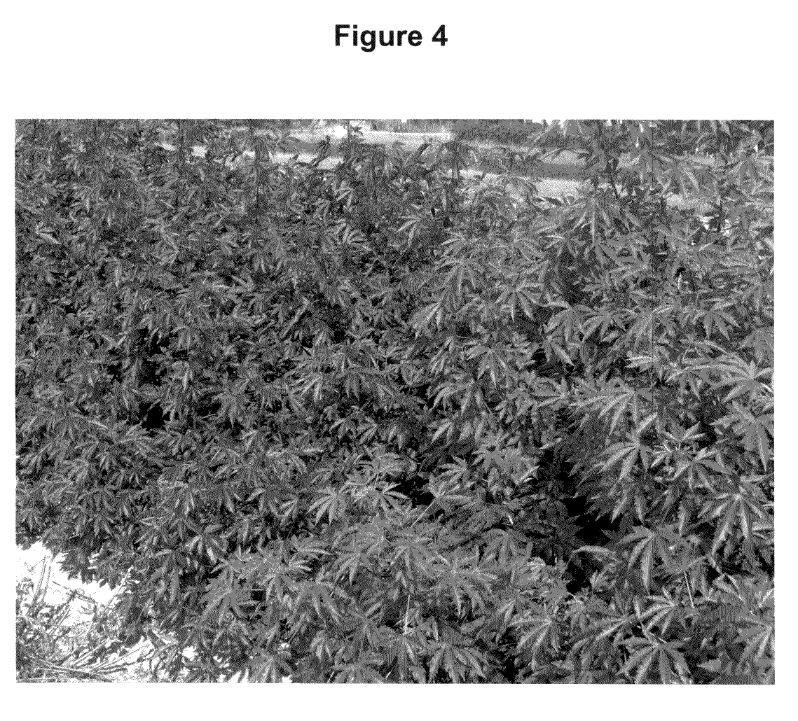

### DETAILED BOTANICAL DESCRIPTION

The following observations, measurements and values describe plants grown in Athens, Ga. at the Athens Select® Test Garden at the University of Georgia Athens campus, by Allan Armitage and Stephanie Anderson. During the growing of the plants, day temperatures ranges from 50° F. to 100° F. and night temperatures ranges from 35° F. to 80° F. In the description, color references are made to The Royal Horticultural Society (RHS) Colour Chart, 1995, Edition, except where general terms of ordinary dictionary meaning are used.
I. Habit: Round to upright
II. Size of plant:
   A. Height.—3-5′ (0.9-1.5 m).
   B. Width.—1-3′ (0.3-0.9 m).
III. Stem:
   A. Color (RHS).—187A.
   B. Length.—2-3′ (0.6-0.9 m).
   C. Diameter.—3-5 mm.
   D. Pubescence.—none.
   E. Shape.—round.
   F. Odor (of bruised stem).—none.
   G. Internode length.—4-5 cm.
IV. Leaf:
   A. Color (RHS).—1. upper 139B 2. lower 147B.
   B. Mature size (L×W).—8.5×8 cm.
   C. Apex.—acuminate.
   D. Base.—truncate.
   E. Margin.—crenate.
   F. Shape.—palmate.
   G. Lobes (present/absent).—1 number 3-5.
   H. Pubescence.—none.
   I. Arrangement on stem.—alternate.
   J. Venation.—pinnipalmate.
   K. Texture.—glabrous.
V. Petiole:
   A. Length.—6-7 cm.
   B. Shape.—round.
   C. Color (RHS).—139B.
   D. Pubescence.—none.
   E. Diameter.—0.3 cm.
VI. Flower:
   A. Inflorescence.—none, solitary 1. number of individual flowers per stem 5-10.
   B. Individual flower.—1. axillary, terminal axillary 2. symmetry radial 3. petals 5 a. size (L×W) 5.75×4.5 cm b. shape spatulate c. apex rounded d. base truncate e. margin entire f. color at peak of bloom 1) upper 187C 2) center 187A 4. pedicels a. color (RHS) 187C b. pubescence none c. length 1-2 mm 5. sepals a. number 5 b. size(L×W) 1.6×0.8 c. shape linear d. pubescence none e. color (RHS) 144B 6. stamens a. number 23-25 b. size (L×W) 0.4×0.8 cm c. color (RHS) 184B d. pollen color (RHS) e. pubescence none 7. pistils a. number 1 b. size of style (L×W) 2.5×0.3 cm c. color of style (RHS) 185B d. color of stigma (RHS) 187A 8. bracts a. number 9-11 b. sixe (L×W) 1.8×0.5 cm c. color (RHS) 126A d. shape spatulate.
   C. Fruit.—no fruit seen.
   D. Seed.—no seed seen.

What is claimed is:

1. A new and distinct variety of Hibiscus plant named ‘Panama Bronze’, substantially as illustrated and described herein.
